(19) United States
(12) Patent Application Publication
(10) Pub. No.: US 2020/0109394 A1
(43) Pub. Date: Apr. 9, 2020

(54) COMPOSITIONS AND METHODS FOR ISOLATING CIRCULATING CELLS

(71) Applicant: Prescient Pharma LLC, Canton, MA (US)

(72) Inventors: Pavel Idelevich, Hingham, MA (US); Arnon Chait, Cleveland, OH (US)

(21) Appl. No.: 16/592,717

(22) Filed: Oct. 3, 2019

Related U.S. Application Data
(60) Provisional application No. 62/740,946, filed on Oct. 3, 2018.

(51) Int. Cl. C12N 13/00; C12N 5/09; G01N 33/50

(52) U.S. Cl. CPC C12N 13/00; G01N 33/5011; C12N 5/0693

(57) ABSTRACT

The invention generally relates to compositions and methods for isolating circulating cells. In one aspect, the invention provides a method for isolating one or more target cells in a biological fluid of a subject, the method comprising: selectively providing to the one or more target cells an effective amount of at least one iron saccharide complex or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field. In further aspects, the method can comprise treating the target cell will one or more cancer treatments. This abstract is intended as a scanning tool for purposes of searching in the particular art and is not intended to be limiting of the present invention.

### CROSS REFERENCE TO RELATED APPLICATIONS

This application claims the benefit of priority to U.S. Provisional Patent Application Ser. No. 62/740,946, filed Oct. 3, 2018, which is hereby incorporated herein by reference in its entirety.

### FIELD OF INVENTION

This disclosure relates to compositions and methods for isolating circulating cells, more specifically, circulating tumor cells (CTCs) and circulating fetal cells (CFCs).

### BACKGROUND

During various diseases or disorders of uncontrolled cellular proliferation, such as cancer, tumor cells may become detached from primary tumors and enter into blood or lymphatic vasculature and travel to other tissues or organs. These detached tumor cells, referred to as circulating tumor cells (CTCs), are believed to be responsible for the spread, or metastasis, of cancer to distant sites in the body. It is estimated that 90% or more of deaths from cancer are from metastasis. Moreover, CTCs are known to have different physical properties compared to neighboring blood cells. Accordingly, there remains a need for selective intracellular delivery methods dependent on the physical properties of these target tumor cells with greater control in delivery activity for diagnostic and/or therapeutic applications. This need and other needs are satisfied by the various aspects of the present disclosure.

### SUMMARY

In accordance with the purpose(s) of the invention, as embodied and broadly described herein, the invention, in one aspect, relates to devices, systems, and methods for isolating one or more circulating cells using iron saccharide complexes and pharmaceutical compositions comprising same. In another aspect, the invention relates to devices, systems, and methods for selectively delivering iron saccharide complexes and pharmaceutical compositions comprising same to one or more circulating cells based on their biophysical properties.

In another aspect, the invention relates to compositions comprising an effective amount of at least one iron saccharide complex for use in detecting, separating, capturing, removing, eliminating, and/or isolating circulating cells, such as, for example circulating tumor cells (CTCs), circulating fetal cells (CFCs), and/or circulating stem cells (CSCs).

In further aspects, disclosed are methods for preventing uncontrolled cellular proliferation (or cell overproliferation) of a target cell, the method comprising the step of selectively providing to the target cell an effective amount of at least one iron saccharide complexes and pharmaceutical compositions comprising same; and isolating the target cell using a magnetic source, thereof, thereby preventing target cell proliferation.

Also disclosed are methods for preventing uncontrolled cellular proliferation (or cell overproliferation) of a target cell, the method comprising the step of selectively providing to the target cell an effective amount of at least one intracellular iron saccharide agent or a pharmaceutically derivative thereof, and magnetically separating the target cells, thereby preventing cellular proliferation of the target cell.

Also disclosed are methods method for the treatment of a target cell in a subject, the method comprising the steps of: diagnosing the subject as having a disorder or disease of uncontrolled cellular proliferation; administering to the subject an effective amount of at least one iron saccharide complex or a pharmaceutically acceptable derivative thereof; and administering to the subject a therapeutically effective amount of one or more cancer treatments.

Also disclosed are kits comprising an effective amount of at least one iron saccharide; and instructions for administering the radiofrequency waves to heat and kill cancer cells, primary tumor and metastasis in connection with the disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 show representative flow cytometry data pertaining to cells incubated with Fe2DG without exposure to magnetic source in accordance with an exemplary embodiment of the present invention.

FIG. 2 show representative flow cytometry data pertaining to cells incubated with Fe2DG after exposure to magnetic source in accordance with an exemplary embodiment of the present invention.

FIG. 3 shows a magnetic source for use in the disclosed methods in accordance with an exemplary embodiment of the present invention.

FIG. 4 shows a tube for use in the disclosed methods in accordance with an exemplary embodiment of the present invention.

FIG. 5 show representative flow cytometry data pertaining to cells incubated with Fe2DG in accordance with an exemplary embodiment of the present invention.

FIG. 6 show representative flow cytometry data pertaining to cells incubated with Fe2DG in accordance with an exemplary embodiment of the present invention.

FIG. 7 show cell stains of HT29 cells (human colon adenocarcinoma) after incubation in medium containing Fe2DG in accordance with an exemplary embodiment of the present invention.

FIG. 8 show cell stains of CRL-1790 cells (human fibroblasts) after incubation in medium containing Fe2DG in accordance with an exemplary embodiment of the present invention.

FIG. 9 show HT29 cell suspension samples incubated with Fe2DG without exposure to a magnetic source.

FIG. 10 show HT29 cell suspension samples incubated with Fe2DG after exposure to a magnetic source in accordance with an exemplary embodiment of the present invention.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In some aspects of the disclosed methods, the subject has been diagnosed with a need for treatment of one or more disorders involving uncontrolled cellular proliferation or cell overproliferation prior to the administering step. In some aspects of the disclosed method, the subject has been diagnosed with a hyperproliferative disorder prior to the administering step. In some aspects of the disclosed method, the subject been diagnosed with a cancer prior to the administering step.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, preventing disorders involving uncontrolled cellular proliferation or cell overproliferation means reducing the incidences, delaying or reversing diseases or disorders that are related to or associated with uncontrolled cellular proliferation or cell overproliferation.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a disorders involving uncontrolled cellular proliferation or cell overproliferation prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the term “providing” refers to any method of administering or contacting a disclosed compound or composition to a cell, target cell, target receptor, or other biological entity. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “pharmaceutically acceptable derivative” refers to any homolog, analog, or fragment corresponding to the disclosed compounds which can modulate or facilitate iron uptake, for example, in the target cell.

The term “modulate” or “modulating” refers to the ability of an agent to regulate a desired response, e.g., inhibiting cellular proliferation including cell killing. Modulate, as used herein, can refer to a process by which an agent elevates or reduces, or increases or decreases, a desired response. Modulate refers to the ability of an agent to regulate a response either directly or indirectly. Modulate can refer to a process by which an agent substantially inhibits, stabilizes, or prevents a response when a response would otherwise increase. Modulate can also refer to a process by which an agent substantially stabilizes, enhances, or maintains a response when a response would otherwise decrease. Thus, compounds disclosed herein as intracellular iron modulators, can function as enhancing agents, for example, to increase intracellular iron concentration of target cells.

As used herein, the term “cancer” means any condition characterized by cells displaying uncontrolled growth, invasion of normal tissue, and/or metastasis.

Compounds described herein may comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., Almarasson, O., et. al. (2004) The Royal Society of Chemistry, 1889-1896. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds and compositions for use in detecting, separating, capturing, removing, eliminating, and/or isolating circulating cells, such as, for example circulating tumor cells (CTCs), circulating fetal cells (CFCs), and/or circulating stem cells (CSCs). In a further aspect, the invention relates to compounds and compositions for use as agents in the treatment of diseases and disorders involving uncontrolled cellular proliferation (or cell overproliferation), such as cancer. More specifically, in one aspect, the present invention relates to compounds and compositions for the treatment or prevention of tumorigenesis and/or malignant cancer development. In further aspects, the disclosed compounds modulate the intracellular iron concentration.

It is contemplated that each disclosed derivative can be optionally further substituted. It is also contemplated that any one or more derivative can be optionally omitted from the invention. It is understood that the disclosed compounds and compositions can be employed in the disclosed methods of using.

In one aspect, the invention relates to iron (III) saccharide complexes or a pharmaceutically acceptable derivate thereof. In further aspects, the saccharide of the least one iron saccharide complex or a pharmaceutically acceptable derivative thereof can comprise a monosaccharide, disaccharide, or polysaccharide, or a combination thereof. In still further aspects, the saccharide of the least one iron saccharide complex or a pharmaceutically acceptable derivative thereof can comprise at least one of: glucose, dextrose, 2-deoxyglucose, thioglucose, galactose, fructose, dextran, dextrin, maltose, arabinose, rhamnose, mannose, sorbose, xylose, lactose, sucrose and raffmose. In yet further aspects, the least one iron saccharide complex or a pharmaceutically acceptable derivative thereof comprises at least one of: iron glucose, iron 2-deoxyglucose, iron thioglucose, iron sucrose, iron dextran, iron fructose, polysaccharide iron complex (PIC) or the like.

In a still further aspect, the iron (III) saccharide complex, or pharmaceutically acceptable derivative thereof is selected from iron glucose, iron 2-deoxyglucose, and iron thioglucose.

In various aspects, the disclosed compounds have specific cytoactivity on a target cell. In a further aspect, the target cell is a circulating tumor cell (CTC), circulating fetal cell (CFC), and/or circulating stem cell (CSC). In a still further aspect, the target cell is a mammalian cell. In a yet further aspect, the mammalian cell is human.

In further aspects, the disclosed compounds can have cancer cell specific cytoactivity, and thus can be effective at treating one or more cancers and/or related disorders. In even further aspects, the disclosed compounds are cytoactive against at least one cancer cell line. In a yet further aspect, the cancer cell line is a leukemia cell. In a still further aspect, the cancer cell line is a lymphoma cell line. In various further aspects, the disclosed compounds can be efficacious against a variety of cancer cell types and cell lines derived from cancer cells.

In a further aspect, the disclosed compounds have cytoactivity on a cell associated with uncontrolled cellular proliferation. In a yet further aspect, the cell associated with uncontrolled cellular proliferation is a cancer cell. In an even further aspect, the cell is a cell cultured in vitro. In a still further aspect, the cell is in vivo.

### Methods of Making the Compounds

The compounds of this invention can be prepared by employing shown reactions, in addition to other standard manipulations that are known in the literature, exemplified in the experimental sections or clear to one skilled in the art. The following examples are provided so that the invention might be more fully understood, are illustrative only, and should not be construed as limiting. For clarity, examples having a fewer substituent can be shown where multiple substituents are allowed under the definitions disclosed herein.

It is contemplated that each disclosed method can further comprise additional steps, manipulations, and/or components. It is also contemplated that any one or more step, manipulation, and/or component can be optionally omitted from the invention. It is understood that a disclosed method can be used to provide the disclosed compounds. It is also understood that the products of the disclosed methods can be employed in the disclosed compositions, methods, kits, and uses.

In further aspects, an exemplary synthesis of Fe (III)-2 deoxy D glucose (Fe2DG) complex compound can be as follows: 2 deoxy D glucose (C6O12O5) (9 mmol, 1.35 g) can be dissolved in 60 mL methanol to which sodium (0.414 g, 18 mmol) can be added in small pieces. To this, 40 mL of methanolic solution of ferric chloride (0.486 g, 3 mmol) can be added to give a yellow precipitate. After stirring for 3 h, a brown colored solid can be obtained. The reaction mixture can be stirred for 24 h in order to ensure the completion of the reaction. The solid can be obtained by filtering the reaction mixture and further washing with ether and drying under vacuum. An isolated product can be purified by dissolving it in a 2:1 water-methanol mixture and can be reprecipitated with ethanol. The purification process can be repeated three times. The product can then be washed with ether and dried under vacuum.

### C. Compositions

In one aspect, the invention relates to compositions comprising an effective amount of at least one iron (III) saccharide complex or a pharmaceutically acceptable derivate thereof. In some aspects, the composition can comprise pharmaceutical compositions. In other aspects, the composition can comprise nutraceutical compositions.

The compounds have cytoactivity on target cells, and generally have IC₅₀ values ranging from about 0.01 micromolar to about 100 millimolar. IC₅₀ refers to the concentration of the compound that is required for 50% antagonism or inhibition of the target in vitro. IC₅₀ also refers to the concentration of a substance that is required for 50% antagonism or inhibition of the target in vivo. The activity of the compounds, including IC₅₀, is determined according to the procedures discussed below in the Examples section.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., Ansel, H. et. al., Pharmaceutical Dosage Forms and Drug Delivery Systems (6th Ed. 1995) at pp. 196 and 1456-1457.

The agents and pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical and nutraceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical and nutraceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the effective amount is a therapeutically effective amount. In a still further aspect, the effective amount is a prophylactically effective amount.

In a further aspect, the composition is formulated for parenteral administration. In a still further aspect, the composition is formulated for inhalation. In yet a further aspect, the composition is formulated for oral administration. In an even further aspect, the composition is formulated for topical administration.

It is understood that the disclosed compositions can be prepared from the disclosed compounds. It is also understood that the disclosed compositions can be employed in the disclosed methods of using.

### D. Methods for Using the Disclosed Compounds and Compositions

### 1. Isolation of Circulating Cells

In various aspects, the disclosed compounds and/or compositions can be useful in detecting, separating, capturing, removing, eliminating, and/or isolating circulating cells, such as, for example circulating tumor cells (CTCs), circulating fetal cells (CFCs), and/or circulating stem cells (CSCs). These targeted circulating cells are known to have different physical properties compared to neighboring blood cells. Thus, in further aspects, the disclosed compounds and/or compositions can be selectively delivered to one or more circulating cells based on their biophysical properties, with greater control in delivery activity for diagnostic and/or therapeutic applications.

In further aspects, the disclosed compounds and/or compositions may be selectively taken up by the target cells. In still further aspects, the uptake in the non-target (healthy) cells occurs less rapidly than in the target (diseased, damaged, and/or pathologically altered) cells. In yet further aspects, the target cells may be tumor cells that have a significantly increased metabolism and, therefore, among other things, show an increased endocytic activity. In even further aspects, cancer cells are known to have a higher glucose uptake and metabolism, and the resulting enhanced glycolysis can serve as an indication of uptake.

In one aspect, disclosed herein is a method for isolating one or more target cells in a biological fluid of a subject, the method comprising: selectively providing to the one or more target cells an effective amount of at least one iron saccharide complex or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field. In further aspects, isolating can comprise passing the biological fluid containing the one or more target cells through a magnetic separation device and/or magnetic filter. In still further aspects, isolating can comprise passing the biological fluid containing the target cells through a microfluidic magnetic separation device.

In further aspects, providing can comprise introducing into the one or more target cell a magnetically responsive amount of the at least one iron saccharide complex. In still further aspects, isolating the one or more target cells can comprise at least one of: contacting the biological fluid with a magnetic separation device, the magnetic separation device comprising a magnetic source and a tube; producing a continuous relative motion between the magnetic source and the biological fluid while the magnetic source produces a magnetic field across the tube, such that magnetically responsive target cells are selectively captured against the tube and wherein the continuous relative motion is applied such that a majority of the biological fluid has access to the magnetic source; and removing all or substantially all the magnetic field produced by the magnetic source across the tube; wherein the magnetically responsive target cells are released from the tube wall.

In further aspects, the method may comprise the step of incubating the target cell in a preparation containing an iron saccharide complex and pharmaceutical compositions comprising same for a predetermined time. During the incubation period, the iron saccharide complex may be selectively taken up by GLUT transporter into the target cells. In still further, it is believed that the uptake in the non-target (healthy) cells occurs less rapidly than in the target (pathologically altered, diseased and/or damaged) cells. In yet further aspects, the target cells may be tumor cells that have a significantly increased metabolism and, therefore, among other things, show an increased endocytic activity. In some aspect, the target cells may be cells that have a significantly increased metabolism and, therefore, among other things, show an increased uptake activity.

### 2. Diagnosis of Isolated Circulating Cells

In various aspects, the method can further comprise performing one or more experiments and/or assays on isolated target cells. In further aspect, the experiment or assay can comprise at least one of identification of a level of a biological marker, cell culture, cell count, cytology analysis, an immunochemical analysis, morphological analysis, genetic analysis, genomics analysis, metabolomics analysis, epigenomics analysis, proteomics analysis, DNA mutation analysis, whole genome analysis, protein and/or RNA expression level of a specific gene or a combination thereof.

In further aspects, the experiment or assay can be for identification of specific mutations. In still further aspects, the identification of specific mutations can be used for treatment decisions and/or guiding therapy choices. In even further aspects, the biological marker is a marker for a disease or disorder of uncontrolled cellular proliferation.

In another aspect, a method of diagnosis comprises performing an experiment upon the isolated target cells and/or subject and identifying a level of a biological marker. In a further aspect, diagnosing comprises determining, in a subject or target cell, levels of a marker (e.g., gene expression) indicative of a state of the subject or patient, the state being predictive as to whether the subject or patient will manifest reduced symptoms in response to a treatment.

In a further aspect, the biological marker is a marker for a disease or disorder of uncontrolled cellular proliferation. In a still further aspect, the subject is a biological fluid or sample. In a still further aspect, the biological fluid or sample is selected from at least one of a cell, blood, saliva, urine, tissue, and phlegm.

In one aspect, diagnosis of a disease or disorder of uncontrolled cellular proliferation comprises a medical history. In a further aspect, the diagnosis comprises comparing the findings of the medical history with the diagnostic standards. In a still further aspect, the diagnosis comprises finding of at least one risk factor for developing a disease or disorder of uncontrolled cellular proliferation. In a yet further aspect, the disease or disorder of uncontrolled cellular proliferation is associated with an abnormal proliferative disease, a degenerative disease, or a cell overproliferation disorder.

### 3. Treatment of a Disorder

In further aspects, the disclosed compounds and/or compositions can be useful in diagnosing, treating and/or preventing diseases and disorders involving uncontrolled cellular proliferation or cell overproliferation that include but are not limited to cancers, premalignant conditions (e.g., hyperplasia, metaplasia, and dysplasia), benign tumors, hyperproliferative disorders, and benign dysproliferative disorders. Cancer is characterized primarily by an increase in the number of abnormal circulating cells derived from a given normal tissue, invasion of adjacent tissues by these abnormal circulating cells, and lymphatic or blood-borne. Malignancies and related disorders that can be treated, prevented, managed, ameliorated, particularly metastatic cancer, by administration of compounds and/or compositions of the invention and isolating the target cell as discussed herein. In further aspects, the invention can comprise treating the one or more isolated target cell and/or subject with one or more cancer treatments as disclosed herein.

Thus, in one aspect, the invention relates to a method of treating a disorder of uncontrolled cellular proliferation of a target cell in a subject, comprising the step of administering to the subject a therapeutically effective amount of one or more cancer treatments as disclosed herein.

In another aspect, the invention relates to a method for inhibiting cell replication in one or more target cells, comprising the step of contacting one or more target cells with an effective amount of an effective amount of at least one iron saccharide complex compound or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field.

In further aspects, the target cell has been isolated from a human prior to the contacting step. In a still further aspect, the step of contacting occurs in vitro. In a yet further aspect, the step of contacting occurs in vivo.

In further aspects, the step of contacting occurs via administration of the compound to a subject. In a still further aspect, the step of contacting occurs via administration of the compound to a subject, wherein the subject is a mammal. In a yet further aspect, the step of contacting occurs via administration of the compound to a subject, wherein the subject is a human. In an even further aspect, the step of contacting occurs via administration of the compound to a subject, wherein the subject is a mouse.

In further aspects, the step of contacting occurs via administration of the compound to a subject, wherein the administration occurs topically, parenterally, orally, intravenously, intramuscularly, subcutaneously, or by aerosol. In a still further aspect, the step of contacting occurs via administration of the compound to a subject, wherein the administration occurs orally, parenterally, intramuscularly, or intravenously. In a yet further aspect, the step of contacting occurs via administration of the compound to a subject, wherein the administration occurs orally. In an even further aspect, the step of contacting occurs via administration of the compound to a subject, wherein the administration occurs intravenously.

In further aspects, the step of contacting occurs via administration of the compound to a subject, wherein the subject has been diagnosed with a need for inhibiting cell replication prior to the administering step. In a still further aspect, the step of contacting occurs via administration of the compound to a subject, wherein the subject has been diagnosed with a need for treatment of a disorder of uncontrolled cellular proliferation prior to the administering step.

In further aspects, contacting the target cell results in an accumulation of iron. In a still further aspect, the amount of iron is magnetically responsive. In a yet further aspect, the amount of iron accumulation is effective to allow the target cells to be magnetically separated from non-target cells, such as healthy cells.

In a further aspect, the target cell is a mammalian cell. In a still further aspect, the mammalian cell is human. In a yet further aspect, the mammalian cell is murine. In an even further aspect, the target cell is a cancer cell.

According to various aspects of the present invention, the disclosed compounds and/or compositions can be useful for the treatment of a cancer, including, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Lymphoma, Hodgkin's disease, non-Hodgkin's disease, multiple myeloma, Waldenstrom's macroglobulinemia, heavy chain disease, Solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, cancers and related disorders that can be treated or prevented by methods and compositions disclosed herein include but are not limited to the following: leukemia, including, but not limited to, acute leukemia, acute lymphocytic leukemia; acute myelocytic leukemia, including, but not limited to, myeloblastic, promyelocytic, myelomonocytic, monocytic, erythroleukemia and myelodysplastic syndrome; chronic leukemia, including, but not limited to, chronic myelocytic (granulocytic) leukemia, chronic lymphocytic leukemia, hairy cell leukemia; polycythemia vera; lymphomas, including, but not limited to, Hodgkin's lymphoma, non-Hodgkin's lymphoma; myeloma, including, but not limited, to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumor, including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer, including, but not limited to, papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers, including, but not limited to, Cushing's disease, prolactin-secreting tumor, acromegaly, and diabetes insipius; eye cancer, including, but not limited to, ocular melanoma such as iris melanoma, choroidal melanoma, and cilliary body melanoma, and retinoblastoma; vaginal cancer, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancer, including, but not limited to, squamous cell carcinoma, and adenocarcinoma; uterine cancer, including, but not limited to, endometrial carcinoma and uterine sarcoma; ovarian cancers, including, but not limited to, ovarian epithelial carcinoma, borderline tumor, germ cell tumor, and stromal tumor; esophageal cancer, including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancer, including, but not limited to, adenocarcinoma, fungating (polypoid), ulcerating, superficial spreading, diffusely spreading, malignant lymphom, liposarcoma, fibrosarcoma, and carcinosarcoma; colon cancer; rectal cancer; liver cancer, including, but not limited to, hepatocellular carcinoma and hepatoblastoma, gallbladder cancer, including, but not limited to, adenocarcinoma; cholangiocarcinoma, including, but not limited to, pappillary, nodular, and diffuse; lung cancer, including, but not limited to, non-small cell lung cancer, squamous cell carcinoma (epidermoid carcinoma), adenocarcinoma, large-cell carcinoma and small-cell lung cancer; testicular cancer, including, but not limited to, germinal tumor, seminoma, anaplastic, classic (typical), spermatocytic, nonseminoma, embryonal carcinoma, teratoma carcinoma, and choriocarcinoma (yolk-sac tumor); prostate cancer, including, but not limited to, adenocarcinoma, leiomyosarcoma, and rhabdomyosarcoma; penal cancers; oral cancer, including, but not limited to, squamous cell carcinoma; basal cancers; salivary gland cancer, including, but not limited to, adenocarcinoma, mucoepidermoid carcinoma, and adenoidcystic carcinoma; pharynx cancer, including, but not limited to, squamous cell cancer, and verrucous; skin cancer, including, but not limited to, basal cell carcinoma, squamous cell carcinoma and melanoma, superficial spreading melanoma, nodular melanoma, lentigo malignant melanoma, and acral lentiginous melanoma; kidney cancer, including, but not limited to, renal cell cancer, adenocarcinoma, hypernephroma, fibrosarcoma, and transitional cell cancer (renal pelvis and/or uterer); Wilms' tumor; bladder cancer, including, but not limited to, transitional cell carcinoma, squamous cell cancer, adenocarcinoma, carcinosarcoma. In addition, cancer includes myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In one aspect, the invention relates to a method for inhibiting cell replication in one or more target cells in a subject, comprising the step of contacting one or more target cells with an effective of at least one iron saccharide complex or a pharmaceutically acceptable derivative thereof; and isolating the target cells using a magnetic source capable of generating a magnetic field effective to capture target cells containing an intracellular concentration of the of at least one iron saccharide complex or a pharmaceutically acceptable derivative thereof in the magnetic field. In a further aspect, inhibiting is preventing. In a still further aspect, the target cell is mammalian. In a yet further aspect, the mammalian cell is human. In an even further aspect, the mammalian cell is murine. In a still further aspect, the target cell is a cancer cell.

In a further aspect, the cancer cell is associated with a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer cell is associated with a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer cell is associated with a cancer selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is triple negative breast cancer. In a still further aspect, the cancer is a myc driven or c-myc associated cancers. In a yet further aspect, the cancer is selected from breast cancer, prostate cancer, colon cancer, ovarian cancer, hepatocellular carcinoma, small cell lung cancer, and non-small cell lung cancer. In an even further aspect, the cancer is a pediatric cancer. In a still further aspect, the cancer is pediatric neuroblastoma.

In a further aspect, the cancer cell is associated with a hematological cancer. In a still further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

In a further aspect, the hematological cancer is leukemia. In a still further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the hematological cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sezary Syndrome, heavy chain disease, and Waldenström macroglobulinemia. In a yet further aspect, the lymphoma is selected from Hodgkin's lymphoma and non-Hodgkin's lymphoma.

In a further aspect, the lymphoma is Hodgkin's lymphoma. In a still further aspect, the Hodgkin's lymphoma is classic Hodgkin lymphoma. In a yet further aspect, the classic Hodgkin's lymphoma is selected from nodular sclerosis Hodgkin lymphoma, mixed cellularity Hodgkin lymphoma, lymphocyte-depleted Hodgkin lymphoma, and Lymphocyte-rich classic Hodgkin lymphoma. In an even further aspect, the Hodgkin's lymphoma is nodular lymphocyte-predominant lymphoma.

In a further aspect, the lymphoma is non-Hodgkin's lymphoma. In a still further aspect, the non-Hodgkin's lymphoma is a B-cell type. In a yet further aspect, the B-cell type non-Hodgkin's lymphoma is selected from Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. In an even further aspect, the non-Hodgkin's lymphoma is a T-cell type. In a still further aspect, the T-cell type non-Hodgkin's lymphoma is selected from mycosis fungoides and the Sézary Syndrome, anaplastic large cell lymphoma, and precursor T-lymphoblastic lymphoma. In a yet further aspect, the non-Hodgkin's lymphoma is a NK-cell type.

In a further aspect, the hematological cancer is a chronic myeloproliferative disorder. In a still further aspect, the chronic myeloproliferative disorder is selected from chronic myelogenous leukemia, polycythemia vera, primary myelofibrosis, chronic idiopathic myelofibrosis, essential thrombocythemia, chronic neutrophilic leukemia, and chronic eosinophilic leukemia.

In a further aspect, the hematological cancer is a myeloplastic syndrome. In a still further aspect, the myeloplastic syndrome is selected from refractory anemia (RA), refractory anemia with ring sideroblasts (RARS), refractory anemia with excess blasts (RAEB), refractory cytopenia with multilineage dysplasia (RCMD), refractory cytopenia with unilineage dysplasia (RCUD), unclassifiable myelodysplastic syndrome (MDS-u), and myelodysplastic syndrome associated with an isolated del(5q).

In a further aspect, the hematological cancer is a myeloproliferative neoplasm. In a still further aspect, the myeloproliferative neoplasm is selected from chronic myelomonocytic leukemia (CMML), juvenile myelomonocytic leukemia (JMML), atypical chronic myeloid leukemia (aCML), and unclassifiable myelodysplastic/myeloproliferative neoplasm.

In a further aspect, the hematological cancer is a plasma cell neoplasm. In a still further aspect, the plasma cell neoplasm is selected from monoclonal gammopathy of undetermined significance (MGUS), isolated plasmacytoma of the bone, extramedullary plasmacytoma, and multiple myeloma.

In a further aspect, the target cell has been isolated from a human prior to a contacting step. In a still further aspect, the step of contacting occurs in vitro. In a yet further aspect, the step of contacting occurs in vivo.

In a further aspect, the step of contacting occurs via administration of the compound to a subject. In a still further aspect, the step of contacting occurs via administration of the compound to a subject, wherein the subject is a mammal. In a yet further aspect, the step of contacting occurs via administration of the compound to a subject, wherein the subject is a human. In an even further aspect, the step of contacting occurs via administration of the compound to a subject, wherein the subject is a mouse.

In a further aspect, the step of contacting occurs via administration of the compound to a subject, wherein the administration occurs topically, parenterally, orally, intravenously, intramuscularly, subcutaneously, or by aerosol. In a still further aspect, the step of contacting occurs via administration of the compound to a subject, wherein the administration occurs orally, parenterally, intramuscularly, or intravenously. In a yet further aspect, the step of contacting occurs via administration of the compound to a subject, wherein the administration occurs orally. In an even further aspect, the step of contacting occurs via administration of the compound to a subject, wherein the administration occurs intravenously.

In a further aspect, the step of contacting occurs via administration of the compound to a subject, wherein the subject has been diagnosed with a need for inhibiting cell replication prior to the administering step. In a still further aspect, the step of contacting occurs via administration of the compound to a subject, wherein the subject has been diagnosed with a need for treatment of a disorder of uncontrolled cellular proliferation prior to the administering step.

Toxicity and therapeutic efficacy of the disclosed compounds can be determined by standard pharmaceutical procedures in cell cultures or experimental animals, e. g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices can be desirable. While compounds that exhibit toxic side effects can be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage for use in humans. The dosage of such compounds lies preferably within a range of circulating concentrations that include the ED50 with little or no toxicity. Dosages can vary within this range depending upon the dosage form employed and the route of administration utilized. For any compound used in disclosed herein, the therapeutically effective dose can be estimated initially from cell culture assays. A dose can be formulated in animal models to achieve a circulating plasma concentration range that includes the IC50 as determined in cell culture experiments. Such information can be used to more accurately determine useful doses in humans. Levels in plasma can be measured, for example, by high performance liquid chromatography.

Suitable daily doses for the treatment or prevention of a disorder described herein can be readily determined by those skilled in the art. A recommended dose of a compound of a compound disclosed herein can be from about 0.1 mg to about 1000 mg per day, e.g., from about 0.1 to about 500 mg/kg/day, 0.1 to about 250 mg/kg/day, or 0.1 to about 100 mg/kg/day, per kg of body weight, given as a single dose, a single once-a-day dose, or as divided doses throughout a selected time period.

The disclosed protocols and compositions can be tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed.

A lower level of proliferation or survival of the contacted cells can indicate that the therapy can be effective to treat a selected disorder in a subject. Alternatively, instead of culturing cells from a patient, protocols can be screened using cells of a tumor or malignant cell line. Many assays known in the art can be used to assess such survival and/or growth; for example, cell proliferation can be assayed by measuring 3H-thymidine incorporation, by direct cell count, by detecting changes in transcriptional activity of known genes such as proto-oncogenes or cell cycle markers; cell viability can be assessed by trypan blue staining, while differentiation can be assessed visually based on changes in morphology, etc.

Compounds for use in therapy can be tested in suitable animal model systems prior to testing in humans, including but not limited to in rats, mice, chicken, cows, monkeys, rabbits, etc. Further, any assays known to those skilled in the art can be used to evaluate the prophylactic and/or therapeutic utility of the combinatorial therapies disclosed herein for treatment, prophylaxis, management or amelioration of one or more symptoms associated with the disease or disorder as described hereinabove.

In a further aspect, the dose is from about 0.001 to about 10000 μM in serum medium. In a still further aspect, the effective amount of the lithium compound is from about 0.01 μM to about 100 μM in serum medium, including exemplary subranges of about 0.1 μM to about 10 μM, about 0.2 μM to about 1 μM, about 0.1 to about 1 μM, about 1 μM to about 100 μM, about 0.1 μM to about 0.5 μM, and about 1 μM to about 50 μM.

In further aspects, the dose can be administered systemically to target cells, such as CTC, primary tumor cells, and/or metastasized tumor cells, followed with application of radiofrequency waves to heat the target cells, and thus eventually selectively killing the target cells (i.e., cancer cells) and potentially curing a subject.

### 4. Co-Therapeutic Use

In various aspects, other cancer treatments can be used in combination with the methods and compounds disclosed herein. In further aspects, such cancer treatment may be administered to isolated target cells and/or subjects. In still further aspects, such treatments include the use of one or more molecules, or compounds for the treatment of cancer (i.e., cancer therapeutics). Some examples include, but are not limited to, chemoagents, immunotherapeutics, cancer vaccines, anti-angiogenic agents, cytokines, hormone therapies, gene therapies, biological therapies, and radiotherapies. While maintaining or enhancing efficacy of treatment, preferably the methods of the present invention increase patient compliance, improve therapy and/or reduce unwanted or adverse effects.

In one aspect, the methods of the invention includes the administration of one or more angiogenesis inhibitors such as but not limited to: angiostatin (plasminogen fragment); antiangiogenic antithrombin III; angiozyme; ABT-627; Bay 12-9566; benefin; bevacizumab; BMS-275291; cartilage-derived inhibitor (CDI); CAI; CD59 complement fragment; CEP-7055; Col 3; combretastatin A-4; endostatin (collagen XVIII fragment); fibronectin fragment; Gro-beta; halofuginone; heparinases; heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); marimastat; metalloproteinase inhibitors (TIMPs); 2-methoxyestradiol; MMI 270 (CGS 27023A); MoAb IMC-1C11; neovastat; NM-3; panzem; PI-88; placenta ribonuclease inhibitor; plasminogen activator inhibitor; platelet factor-4 (PF4); prinomastat; prolactin 16 kD fragment; proliferin-related protein (PRP); PTK 787/ZK 222594; retinoids; solimastat; squalamine; SS3304; SU5416; SU6668; SU11248; tetrahydrocortisol-S; tetrathiomolybdate; thalidomide; thrombospondin-1 (TSP-1); TNP-470; transforming growth factor-beta (TGF-b); vasculostatin; vasostatin (calreticulin fragment); ZD6126; ZD 6474; farnesyl transferase inhibitors (FTI); and bisphosphonates.

Additional examples of anti-cancer agents that can be used in the various aspects disclosed herein, including pharmaceutical compositions and dosage forms disclosed herein, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. These two agents are particularly useful when used in methods employing thalidomide and a topoisomerase inhibitor.

In a further aspect, the treatment methods disclosed herein includes the administration of one or more immunotherapeutic agents, such as antibodies and immunomodulators, which include, but are not limited to, HERCEPTIN, RITUXAN, OVAREX™, PANOREX@, BEC2, IMC-C225, VITAMIN, CAMPATH@ I/H, Smart MI95, LYMPHOCIDE™, Smart ID10, and ONCOLYM™, rituximab, gemtuzumab, or trastuzumab.

In a still further aspect, the treatment methods disclosed herein includes administering one or more anti-angiogenic agents, which include, but are not limited to, angiostatin, thalidomide, kringle 5, endostatin, other Serpins, anti-thrombin, 29 kDa N-terminal and 40 kDa C-terminal proteolytic fragments of fibronectin, 16 kDa proteolytic fragment of prolactin, 7.8 kDa proteolytic fragment of platelet factor-4, a 13-amino acid peptide corresponding to a fragment of platelet factor-4 (Maione et al., 1990, Cancer Res. 51: 2077), a 14-amino acid peptide corresponding to a fragment of collagen I (Tolma et al., 1993, J. Cell Biol. 122: 497), a 19 amino acid peptide corresponding to a fragment of Thrombospondin I (Tolsma et al., 1993, J Cell Biol. 122: 497), a 20-amino acid peptide corresponding to a fragment of SPARC (Sage et al., 1995, J: Cell. Biochem. 57: 1329-), or any fragments, family members, or derivatives thereof, including pharmaceutically acceptable derivatives thereof.

In one aspect, the treatment methods disclosed herein can comprise the use of radio waves, infrared, and/or radiation. In a further aspect, the treatment methods disclosed herein can comprise the use of thermal ablation.

In a further aspect, the treatment methods further comprises the administration of one or more cytokines, which includes, but is not limited to, lymphokines, tumor necrosis factors, tumor necrosis factor-like cytokines, lymphotoxin-α, lymphotoxin-β, interferon-α, interferon-β, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40 or CD137 ligands, Fas-Fas ligand, 4-1BBL, endothelial monocyte activating protein or any fragments, family members, or derivatives thereof, including pharmaceutically acceptable salts thereof.

In a further aspect, the treatment method comprises hormonal treatment. Hormonal therapeutic treatments comprise hormonal agonists, hormonal antagonists (e.g., flutamide, tamoxifen, leuprolide acetate (LUPRON™), LH-RH antagonists), inhibitors of hormone biosynthesis and processing, steroids (e. g., dexamethasone, retinoids, betamethasone, cortisol, cortisone, prednisone, dehydrotestosterone, glucocorticoids, mineralocorticoids, estrogen, testosterone, progestins), antigestagens (e. g., mifepristone, onapristone), antiandrogens (e. g., cyproterone acetate), and the like.

In a further aspect, the disclosure also relates to kits comprising at least one disclosed compound and one or more other therapeutically active compounds, which are usually applied in the treatment of the above-mentioned conditions. For example, the disclosed kits can comprise therapeutically effective amounts of one or more disclosed compound and one or anti-cancer agents. The kits can be co-packaged, co-formulated, and/or co-delivered with the anti-cancer agents. For example, a drug manufacturer, a drug reseller, a physician, or a pharmacist can provide a disclosed kit for delivery to a patient.

In a further aspect, the disclosed compounds, compositions, and methods can be used prophylactically, i.e., to prevent progression to a neoplastic or malignant state, including but not limited to those disorders listed above. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79.) Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

In another aspect, the invention also relates to methods for preventing hyperproliferative cellular activity in a subject, the method comprising the step of providing to the subject an effective amount of at least one iron saccharide complex for intracellular uptake; and administering x-ray or radiation effective to cause an intracellular action of the iron agent. In a further aspect, the intracellular action comprises potentiation (increase) of damaging effect of radiation from iron accumulated in the target cells (e.g., cancer cells, tumor cells, and metastasis). In a still further aspect, the present methods can allow more effective and more targeted way to kill target cells, for example cancer cells, using radiotherapy. In a yet further aspect, the step of providing to the subject comprises providing to the subject an effective amount of radio waves and/or infrared to heat iron accumulated in target cells.

The effective amount or dosage of the compounds can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 1 mg to about 10,000 mg, preferably from about 2 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days. In some aspects, the compounds can be administered on a regimen of 1 to 4 times per day, preferably once or twice per day. This dosing regimen can be adjusted to provide the optimal therapeutic response.

In determining the effective dose or dosage of the pharmaceutical composition of the invention, a response to a prophylactic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the diagnostic methods that are used to ascertain the likelihood that a subject has a hyperproliferative disorder or disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount can depend upon the degree to which an individual has abnormal levels and/or activity of a pathway associated protein or pathway associated protein complex.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

In some aspects, the effective amount is a therapeutically effective amount. In other aspects, the effective amount is a prophylactically effective amount. In further aspects, the subject is a mammal. In still further aspects, the mammal is a human.

In a further aspect, the method further comprises the step of identifying a subject in need of anti-hyperproliferative treatment. In a still further aspect, the subject in need of anti-hyperproliferative treatment comprises having at least one risk factor for developing a hyperproliferative disease or disorder.

### E. Kits

In one aspect, the invention relates to a kit comprising an effective amount of at least one iron saccharide complex compound or a pharmaceutically acceptable derivative thereof, and one or more of: (a) instructions the iron saccharide complex in connection with isolating one or target cells associated with a disorder or disease of uncontrolled cellular proliferation using; (b) at least one agent known to treat the disorder or disease of uncontrolled cellular proliferation; and (c) instructions for treating the disorder or disease of uncontrolled cellular proliferation.

In a further aspect, the at least one agent is a hormone therapy agent. In a still further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the at least one agent is a chemotherapeutic agent. In a still further aspect, the chemotherapeutic agent is selected from one or more of the group consisting of an alkylating-like agent, an antimetabolite agent, an antineoplastic antibiotic agent, a mitotic inhibitor agent, an mTor inhibitor agent or other chemotherapeutic agent.

In a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

In a further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In a further aspect, the alkylating-like agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

In a further aspect, the mitotic inhibitor agent is selected from one or more of the group consisting of etopside, vincristine, ixabepilone, vinorelbine, vinblastine, and teniposide, or a pharmaceutically acceptable salt, hydrate, solvate, or polymorph thereof.

In a further aspect, the mTor inhibitor agent is selected from one or more of the group consisting of everolimus, siroliumus, and temsirolimus, or a pharmaceutically acceptable salt, hydrate, solvate, or polymorph thereof.

In a further aspect, the kit further comprises instructions to provide the compound in connection with surgery. In a still further aspect, the kit further comprises instructions to provide the compound in connection with surgery, wherein the instructions provide that surgery is performed prior to the administering of at least one compound. In a yet further aspect, the kit further comprises instructions to provide the compound in connection with surgery, wherein the instructions provide that surgery is performed after the administering of at least one compound. In an even further aspect, the kit further comprises instructions to provide the compound in connection with surgery, wherein the instructions provide that surgery is performed after the administering of at least one compound, and wherein the instructions provide that the administering of at least one compound is to effect presurgical removal of circulating tumor cells. In a still further aspect, the kit further comprises instructions to provide the compound in connection with surgery, wherein the instructions provide that surgery is performed after the administering of at least one compound, and wherein the instructions provide that surgery is performed at about the same time as the administering of at least one compound.

In a further aspect, the kit further comprises instructions to provide the compound in connection with radiotherapy and/or thermal ablation. In a still further aspect, the kit further comprises instructions to provide the compound in connection with radiotherapy, wherein the instructions provide that radiotherapy is performed prior to the administering of at least one compound. In a yet further aspect, the kit further comprises instructions to provide the compound in connection with radiotherapy, wherein the instructions provide that radiotherapy is performed after the step of the administering of at least one compound. In an even further aspect, the kit further comprises instructions to provide the compound in connection with radiotherapy, wherein the instructions provide that radiotherapy is performed at about the same time as the step of the administering of at least one compound.

In a further aspect, the kit further comprises a plurality of dosage forms, the plurality comprising one or more doses; wherein each dose comprises a therapeutically effective amount of the compound and the at least one agent. In a still further aspect, the kit further comprises a plurality of dosage forms, the plurality comprising one or more doses; wherein each dose comprises a therapeutically effective amount of the compound and the at least one agent, and wherein each dose of the compound and the at least one agent are co-formulated. In a yet further aspect, the kit further comprises a plurality of dosage forms, the plurality comprising one or more doses; wherein each dose comprises a therapeutically effective amount of the compound and the at least one agent, and wherein each dose of the compound and the at least one agent are co-packaged.

In a further aspect, the kit further comprises a plurality of dosage forms, the plurality comprising one or more doses; wherein each dose comprises a therapeutically effective amount of the compound and the at least one agent, and wherein the dosage forms are formulated for oral administration and/or intravenous administration. In a still further aspect, the kit further comprises a plurality of dosage forms, the plurality comprising one or more doses; wherein each dose comprises a therapeutically effective amount of the compound and the at least one agent, and wherein the dosage forms are formulated for oral administration. In a yet further aspect, the kit further comprises a plurality of dosage forms, the plurality comprising one or more doses; wherein each dose comprises a therapeutically effective amount of the compound and the at least one agent, and wherein the dosage forms are formulated for intravenous administration.

In a further aspect, the kit further comprises a plurality of dosage forms, the plurality comprising one or more doses; wherein each dose comprises a therapeutically effective amount of the compound and the at least one agent; and wherein the dosage form for the compound is formulated for oral administration and the dosage form for the at least one agent is formulated for intravenous administration. In a still further aspect, the kit further comprises a plurality of dosage forms, the plurality comprising one or more doses; wherein each dose comprises a therapeutically effective amount of the compound and the at least one agent; and wherein the dosage form for the compound is formulated for intravenous administration and the dosage form for the at least one agent is formulated for oral administration.

In a further aspect, the instructions for treating a disorder of uncontrolled cellular proliferation provide instructions for treating a cancer. In a still further aspect, the cancer is triple negative breast cancer. In a still further aspect, the cancer is a myc driven or c-myc associated cancers. In a yet further aspect, the cancer is selected from breast cancer, prostate cancer, colon cancer, ovarian cancer, hepatocellular carcinoma, small cell lung cancer, and non-small cell lung cancer. In an even further aspect, the cancer is a pediatric cancer. In a still further aspect, the cancer is pediatric neuroblastoma.

In a further aspect, the cancer is a hematological cancer. In a still further aspect, the hematological cancer is selected from a leukemia, lymphoma, chronic myeloproliferative disorder, myelodysplastic syndrome, myeloproliferative neoplasm, and plasma cell neoplasm (myeloma).

In a further aspect, the cancer is a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer is a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is a hematological cancer. In a still further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

In a further aspect, the hematological cancer is leukemia. In a still further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the hematological cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenström macroglobulinemia. In a yet further aspect, the lymphoma is selected from Hodgkin's lymphoma and non-Hodgkin's lymphoma.

In a further aspect, the lymphoma is Hodgkin's lymphoma. In a still further aspect, the Hodgkin's lymphoma is classic Hodgkin lymphoma. In a yet further aspect, the classic Hodgkin's lymphoma is selected from nodular sclerosis Hodgkin lymphoma, mixed cellularity Hodgkin lymphoma, lymphocyte-depleted Hodgkin lymphoma, and Lymphocyte-rich classic Hodgkin lymphoma. In an even further aspect, the Hodgkin's lymphoma is nodular lymphocyte-predominant lymphoma.

In a further aspect, the lymphoma is non-Hodgkin's lymphoma. In a still further aspect, the non-Hodgkin's lymphoma is a B-cell type. In a yet further aspect, the B-cell type non-Hodgkin's lymphoma is selected from Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. In an even further aspect, the non-Hodgkin's lymphoma is a T-cell type. In a still further aspect, the T-cell type non-Hodgkin's lymphoma is selected from mycosis fungoides and the Sézary Syndrome, anaplastic large cell lymphoma, and precursor T-lymphoblastic lymphoma. In a yet further aspect, the non-Hodgkin's lymphoma is a NK-cell type.

In a further aspect, the hematological cancer is a chronic myeloproliferative disorder. In a still further aspect, the chronic myeloproliferative disorder is selected from chronic myelogenous leukemia, polycythemia vera, primary myelofibrosis, chronic idiopathic myelofibrosis, essential thrombocythemia, chronic neutrophilic leukemia, and chronic eosinophilic leukemia.

In a further aspect, the hematological cancer is a myeloplastic syndrome. In a still further aspect, the myeloplastic syndrome is selected from refractory anemia (RA), refractory anemia with ring sideroblasts (RARS), refractory anemia with excess blasts (RAEB), refractory cytopenia with multilineage dysplasia (RCMD), refractory cytopenia with unilineage dysplasia (RCUD), unclassifiable myelodysplastic syndrome (MDS-u), and myelodysplastic syndrome associated with an isolated del(5q).

In a further aspect, the hematological cancer is a myeloproliferative neoplasm. In a still further aspect, the myeloproliferative neoplasm is selected from chronic myelomonocytic leukemia (CMML), juvenile myelomonocytic leukemia (JMML), atypical chronic myeloid leukemia (aCML), and unclassifiable myelodysplastic/myeloproliferative neoplasm.

In a further aspect, the hematological cancer is a plasma cell neoplasm. In a still further aspect, the plasma cell neoplasm is selected from monoclonal gammopathy of undetermined significance (MGUS), isolated plasmacytoma of the bone, extramedullary plasmacytoma, and multiple myeloma.

The compounds, compositions, and methods disclosed herein can be useful for the treatment or prevention of one or more disorders involving uncontrolled cellular proliferation (or cell overproliferation) in a subject, as discussed hereinabove. In general, a subject can be any age, including a fetus. A subject to which a compound or compositions disclosed herein can be administered can be an animal, including but not limited to a mammal, such as a non-primate mammal (e.g., cows, pigs, sheep, goats, horses, chickens, dogs, rats, etc.) and a primate (e.g., a monkey such as an acynomolgous monkey and a human) A subject can also be a laboratory animal (e.g., a mouse, rabbit, guinea pig, fruit fly, etc.).

In one aspect, a subject can be diagnosed with one or more disorders as discussed herein elsewhere. In a specific aspect, a subject can be diagnosed with one or more disorders as discussed herein elsewhere before the step of administering to the subject a therapeutically effective amount of one more compounds disclosed herein.

In a further aspect, a subject can be a subject in need of treatment for disorder of uncontrolled cellular proliferation, e.g., cancer. In a still further aspect, a subject can have cancer or a related disorder, as discussed hereinbefore. In one aspect, a subject can be treated prophylactically with a compound or composition disclosed herein, as discussed herein elsewhere.

One or more compounds or compositions disclosed herein can be utilized for the prevention of a variety of cancers, e. g, in individuals who are predisposed as a result of familial history or in individuals with an enhanced risk to cancer due to environmental factors.

The methods and compositions of the invention can be used in patients who are treatment naive, in patients who have previously received or are currently receiving treatment with other pharmaceutical agents or combinations, including but not limited to anti-cancer agents. Other subjects can include patients that have metastasis or no metastasis.

The methods and compositions of the invention are useful not only in untreated patients but are also useful in the treatment of patients partially or completely un-responsive to other treatments. In various aspects, the disclosure provides methods and compositions useful for the treatment of diseases or disorders in patients that have been shown to be or can be refractory or non-responsive to therapies comprising the administration of other agents.

In one aspect, subjects that can be treated with the compositions disclosed herein include those subjects displaying the presence of one or more characteristics of a transformed phenotype, or of a malignant phenotype, displayed in vivo or displayed in vitro by a cell sample from a subject, can indicate the desirability of prophylactic/therapeutic administration of a compound or composition disclosed herein. As mentioned hereinabove, such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 Dalton cell surface protein, etc.

In a further aspect, a subject that exhibits one or more of the following predisposing factors for malignancy can be treated by administration of an effective amount of a compound disclosed herein to remove circulating tumors cells: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome.

It is understood that the disclosed kits can be prepared from the disclosed compounds, products, and pharmaceutical compositions. It is also understood that the disclosed kits can be employed in connection with the disclosed methods of using.

### F. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Specific Depletion of HT29 Cells after Incubation with Fe2Dg by External Magnet from Mixture of these Cells with Whole Blood

In this Example, the effect of external magnet on suspension HT29 cells (human adenocarcinoma) in whole blood after adding of Fe2DG was studied.

For routine maintenance, HT29 cells (ATCC HTB-38) were grown in McCoy's 5A complete growth medium (Cat. No. 30-2007; ATCC, Manassas, Va.) supplemented by 10% FBS, and 1× Penicillin/Streptomycin in an incubator at 37° C. and 5% CO2. Cells were passaged every 4 days.

The cells were detached from flasks using trypsin and re-suspended in 5 mL of fresh McCoy's 5A medium.

Suspension of HT29 cells (ATCC HTB-38) was count by Countess automated cell counter (Invitrogen). Results: total 1.5×106/ml, live cells 1.4×106/ml, dead cells 1.3×105/ml, Viability 91%

Cells were stained by BCECF AM (Life technologies Ref # B1150) intracellular fluorescent dye, according manufacturer instructions. 1 mg of BCECF AM (Invitrogen) was dissolved in 1 ml of DMSO and 10 aliquots were prepared. 1 aliquot was dissolved in 10 ml of Hank's Balanced Salt Solution to make working concentration (0.016 mM). Working solution of BCECF AM was added to HT29 cells suspension (5 ml of BCECF AM 5 ml of HT29 cells suspension in medium) and incubated in room temperature for 30 minutes in the dark.

500 ul of HT29 suspension was added to 4 ml of fresh human volunteer whole blood. Fe2DG in final concentration 1 mg per 1 ml was added. The blood then was incubated in RT for 2 hours.

Then the blood was divided on two portions. Both portions were placed inside of plastic tube (10 cm long, 3 mm outside diameter). One tube was placed inside of the round magnet, another tube was outside of the magnet. After incubation for 30 min; contents of both tubes were gently pushed out. RBC were lysed by adding 4 volumes of distillate water for 30 sec. Then PBS was added to restore osmolarity. After centrifugation for 5 min 1000 rpm and removing of supernatant, the pellet was resuspended in 0.5 ml of PBS and introduced for analysis by BD FACS Aria III

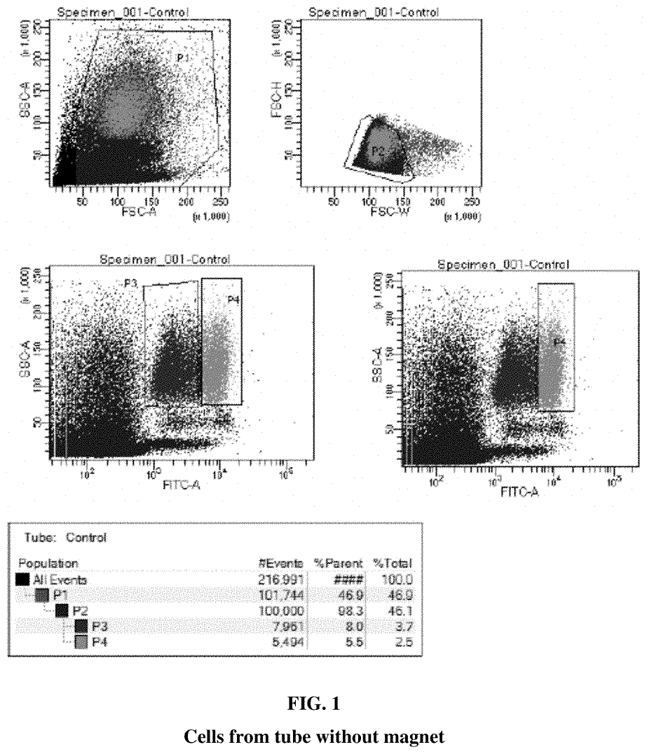

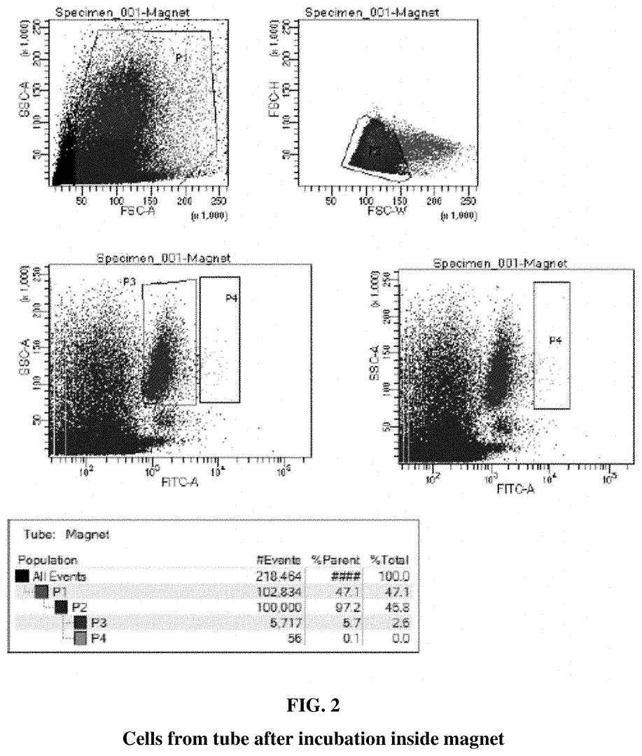

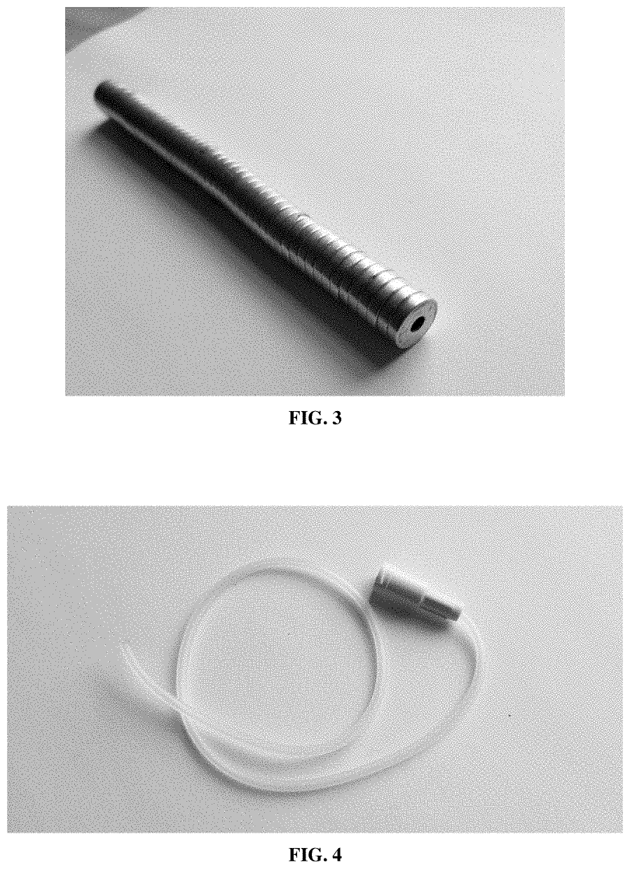

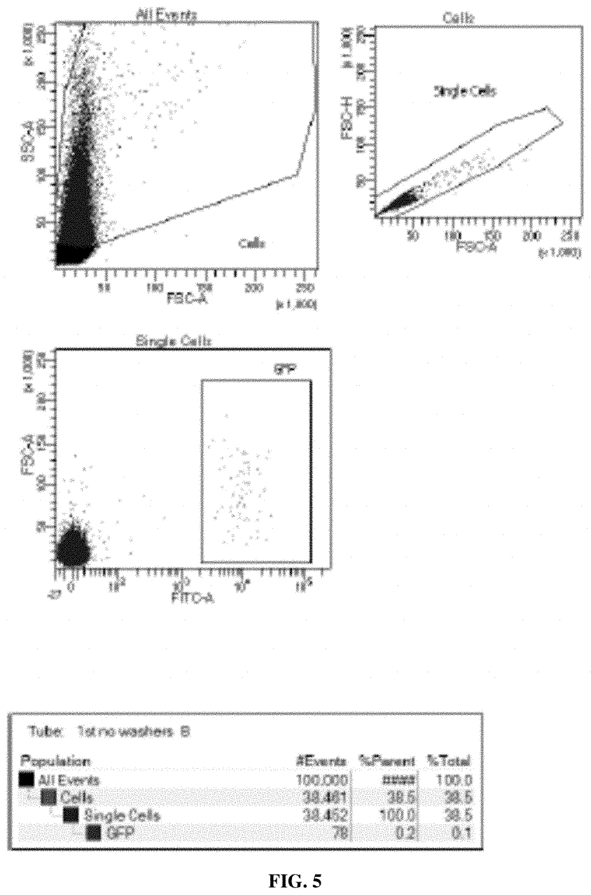

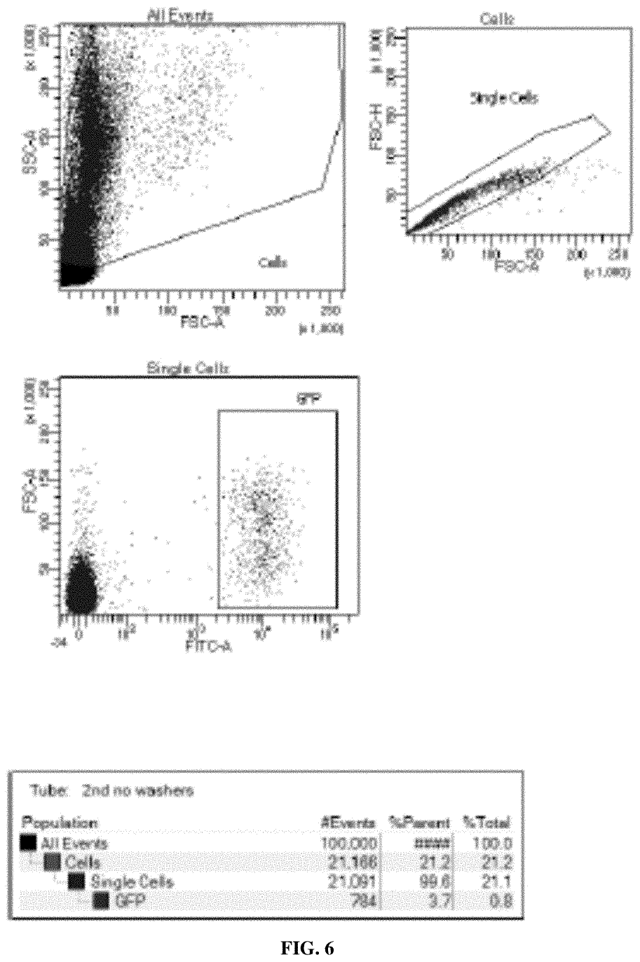

As can be seen from the FIGS. 1 and 2, incubation of the HT29 cell suspension, after loading these cells with Fe2DG, inside of magnet lead to specific (without affect WBC) trapping cancer cells inside of the tube, which manifest by significant depletion of these cells stained by BCECF AM (gate 4) in the sample taking by gentle pushing of the suspension out of the tube.

### 2. Specific Enrichment of A375-EGFP-Puro Cells Incubated with Fe2Dg by External Magnet from Mixture of these Cells with Whole Human Blood

In this second Example, the effect of external magnet on suspension A375-eGFP-puro cells (human melanoma) in whole blood after adding of Fe2DG was studied.

A375-eGFP-puro cells (Imanis Life Science) has enhanced Green Fluorescent Protein as a reporter. And can be detected by fluorescent microscope of FACS because of green fluorescence.

For routine maintenance, A375-eGFP-puro cells were grown in DMEM complete growth medium (Cat. No. 30-2002; ATCC, Manassas, Va.) supplemented by 10% FBS, Penicillin/Streptomycin and 1 ug/ml Puromycin in an incubator at 37° C. and 5% CO₂. Cells were passaged every 4 days. The cells were detached from flasks using trypsin and re-suspended in 5 mL of fresh DMEM medium.

500 ul of A375-eGFP-puro cells suspension was added to 4 ml of fresh human volunteer whole blood. Fe2DG in final concentration 1 mg per 1 ml was added. The blood then was incubated in RT for 2 hours. Then the blood was placed inside of plastic tube (10 cm long, 3 mm outside diameter). The tube was placed inside of the round magnet. After incubation for 30 min; 0.1 ml of the blood was gently pushed through the tube and through the magnet. Collected blood out of the end of the tube was called fraction #1 (1^{st}). Then the tube was taken out of the magnet, and another 0.1 ml was pushed out the tube and was called fraction #2 (2^{nd}). RBC were lysed by adding 4 volumes of distillate water for 30 sec. Then PBS was added to restore osmolarity. After centrifugation for 5 min 1000 rpm and removing of supernatant, the pellet was resuspended in 0.5 ml of PBS and introduced for analysis by BD fluorescence-activated cell sorter (FACS) Aria III.

As can be seen from the FACS data below, it was 18.5 times enrichment of A375-eGFP-puro cells in the second fraction (2^{nd}).

### 3. Prussian Blue Staining (Special Stain for Iron (Fe) of HT29 Cells (Human Colon Adenocarcinoma) and Crl-1790 Cells (Human Fibroblasts) after Incubation in the Medium with Fe2Dg Added

In this third Example, the differential ability of normal vs cancer cells (higher for malignant cells) to accumulate Iron (Fe) from Fe2DG added to medium was studied.

For routine maintenance, HT29 cells (ATCC HTB-38) were grown in McCoy's 5 A complete growth medium (Cat. No. 30-2007; ATCC, Manassas, Va.) supplemented by 10% FBS, and 1× Penicillin/Streptomycin in an incubator at 37° C. and 5% CO₂. Cells were passaged every 4 days.

For Fe2DG treatments, HT29 cells (ATCC HTB-38) cells were grown in 25 cm² Nunclon Delta Surface flasks (Cat. No. 136196; Thermo Fisher Scientific, Waltham, Mass.) with McCoy's 5A complete medium and kept in an incubator at 37° C. and 5% CO₂ until close to 75% confluence was reached.

For routine maintenance, CCD 841 CoN human colon fibroblast cells (CRL-1790; ATCC, Manassas, Va.) were grown in EMEM complete growth medium (Cat. No. 30-2003; ATCC, Manassas, Va.) supplemented by 10% FBS, and 1× Penicillin/Streptomycin in an incubator at 37° C. and 5% CO₂. Cells were passaged every 4 days.

For Fe2DG treatments, CCD 841 CoN cells were grown in 25 cm² Nunclon Delta Surface flasks (Cat. No. 136196; Thermo Fisher Scientific, Waltham, Mass.) with EMEM complete medium and kept in an incubator at 37° C. and 5% CO₂ until close to 75% confluence was reached.

Then medium for both cell types was replaced by DMEM no glucose (ThermoFisher Cat. No. 11966025) supplemented by 10% FBS and Fe2DG 1 mg/ml for two hours.

Then medium was removed and both flasks were stained by Prussian blue staining kit “Advanced testing Iron stain” (ThermoFisher Cat. No. 87006). Cells in the flasks were fixed by 10% Formalin, then rinsed by deionized water. Then the cells were stained by freshly prepared Working Iron Solution 30 minutes at room temperature. Then the cells were rinsed by deionized water and counterstain by Nuclear Red Satin Solution for 30 seconds. Then the cells were rinsed by deionized water and the flasks were examined under microscope (Olympus BX51) and pictures were taken by digital camera (Olympus DP73).

Stain results: Iron—Blue; Background—Red to light Pink

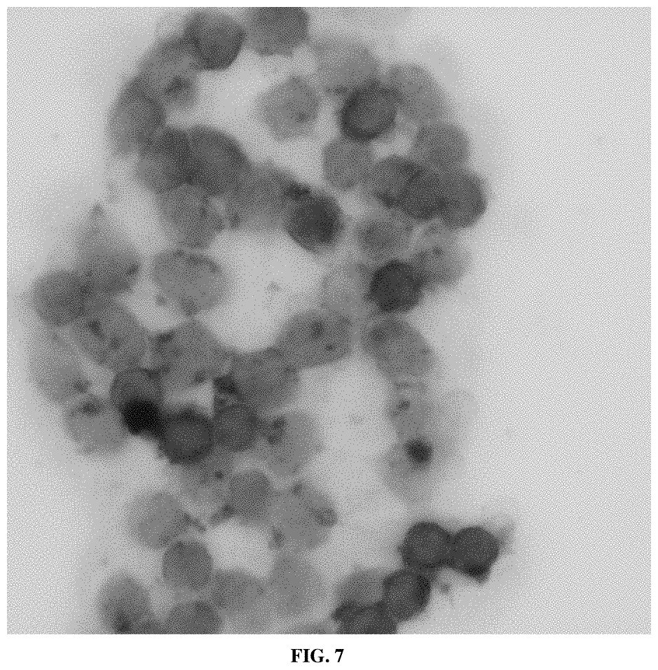

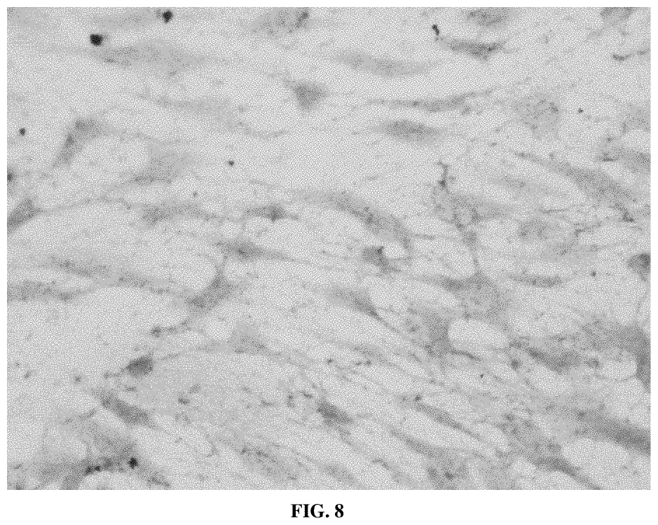

As can be seen from FIGS. 7 and 8, there are significant accumulation of iron in cancer cells (HT29) and practically no positive blue stain (practically no accumulation of Iron) in normal human fibroblasts (CRL-1790).

### 4. Depletion of HT29 Cells Loaded with Fe2Dg by External Magnet

In this fourth Example, the effect of external magnet on suspension HT29 cells (human adenocarcinoma) in complete McCoy's 5A medium after adding of Fe2DG was studied.

Suspension of HT29 cells (ATCC HTB-38) in McCoy's 5A medium with 10% of FBS was count by Countess automated cell counter (Invitrogen). Results: live cells 3.6×106/ml, dead cells 1.6×105/ml, Viability 96%

Fe2DG was added to the cell suspension in final concentration 1 mg per 1 ml. Cells then were incubated in RT for 1 hour. Then the suspension was divided on two portions. Both portions were placed inside of plastic tube (10 cm long, 3 mm in diameter). One tube was placed inside of the round magnet, another tube was outside of the magnet. After incubation for 30 min; contains of both tubes were gently pushed out, and placed on the glass slide, and stained by Hematoxylin. Slides were examined under light microscope Olympus BX51 and pictures were taken using Olympus DP73 digital camera.

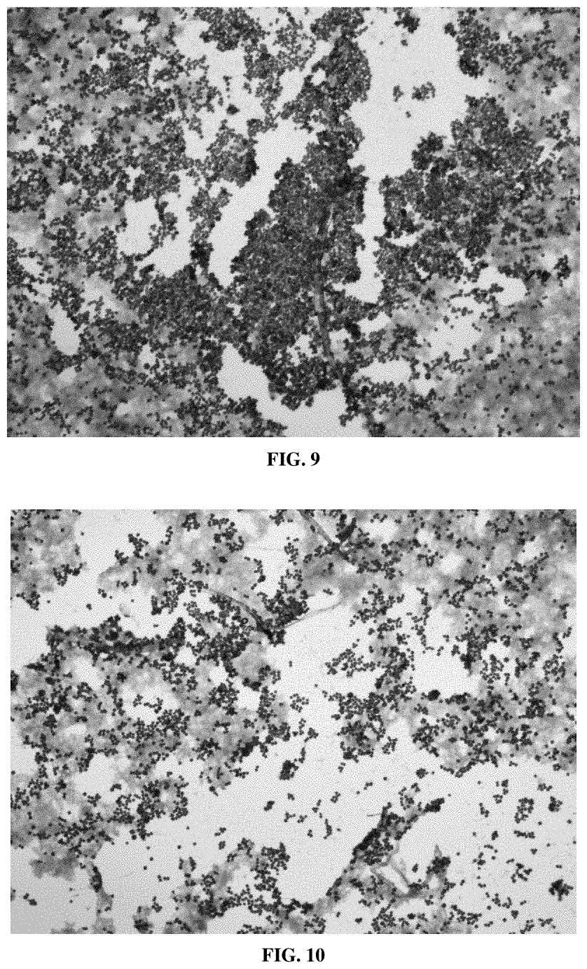

As can be seen from the FIGS. 9 and 10, incubation of the HT29 cell suspension, after loading these cells with Fe2DG, inside of magnet lead to trapping cancer cells inside of the tube, which manifest by significant depletion of these cells in the sample taking by gentle pushing of the suspension out of the tube.

The invention includes at least the following aspects:

Aspect 1: A method for isolating one or more target cells in a biological fluid of a subject, the method comprising: selectively providing to the one or more target cells an effective amount of at least one iron saccharide complex or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field.

Aspect 2: The method of aspect 1, wherein a saccharide of the least one iron saccharide complex or a pharmaceutically acceptable derivative thereof comprises a monosaccharide, disaccharide, or polysaccharide, or a combination thereof.

Aspect 3: The method of any preceding aspect, wherein a saccharide of the least one iron saccharide complex or a pharmaceutically acceptable derivative thereof comprises at least one of: glucose, dextrose, 2-deoxyglucose, thioglucose, galactose, fructose, dextran, dextrin, maltose, arabinose, rhamnose, mannose, sorbose, xylose, lactose, sucrose and raffmose.

Aspect 4: The method of any preceding aspect, wherein the least one iron saccharide complex or a pharmaceutically acceptable derivative thereof comprises at least one of: iron glucose, iron 2-deoxyglucose, iron thioglucose, iron sucrose, iron dextran, iron fructose, polysaccharide iron complex (PIC) or the like.

Aspect 5: The method of any preceding aspect, wherein the one or more target cells are at least one of circulating tumor cells (CTCs), circulating fetal cells (CFCs), and circulating stem cells (CSCs).

Aspect 6: The method of any preceding aspect, wherein isolating comprises passing the biological fluid containing the one or more target cells through a magnetic separation device and/or magnetic filter.

Aspect 7: The method of any preceding aspect, wherein the magnetic filter comprises a venous circulation and/or arterio-venous shunt.

Aspect 8: The method of any preceding aspect, wherein isolating comprises passing the biological fluid containing the target cells through a microfluidic magnetic separation device.

Aspect 9: The method of any preceding aspect, wherein providing comprises introducing into the one or more target cell a magnetically responsive amount of the at least one iron saccharide complex.

Aspect 10: The method of any preceding aspect, wherein providing comprises incubating the target cell in a preparation containing the at least one iron saccharide complex and pharmaceutical compositions comprising same for a predetermined time.

Aspect 11: The method of any preceding aspect, wherein isolating the one or more target cells comprises at least one of: a) contacting the biological fluid with a magnetic separation device, the magnetic separation device comprising a magnetic source and a tube; b) producing a continuous relative motion between the magnetic source and the biological fluid while the magnetic source produces a magnetic field across the tube, such that magnetically responsive target cells are selectively captured against the tube and wherein the continuous relative motion is applied such that a majority of the biological fluid has access to the magnetic source; and c) substantially removing the magnetic field produced by the magnetic source across the tube; wherein the magnetically responsive target cells are released from the tube wall.

Aspect 12: The method of any preceding aspect, wherein the biological fluid comprises at least one of body fluid, whole blood, plasma, any cell-containing blood fraction, cerebrospinal fluid, bone marrow, cell sample, saliva, urine, feces, tissue, or phlegm, or the like.

Aspect 13: The method of any preceding aspect, wherein diagnosing comprises performing a physical examination upon the subject and making a finding.

Aspect 14: The method of any preceding aspect, further comprising performing an experiment or assay on at least one of: the isolated target cells, biological fluid, and the subject.

Aspect 15: The method of any preceding aspect, wherein the experiment or assay comprises at least one of identification of a level of a biological marker, cell culture, an immunochemical analysis, morphological analysis, genomics analysis, metabolomic-s, epigenomics analysis, proteomics analysis, DNA mutation analysis, whole genome analysis, protein and/or RNA expression level of a specific gene or a combination thereof.

Aspect 16: The method of any preceding aspect, wherein the biological marker is a marker for a disease or disorder of uncontrolled cellular proliferation.

Aspect 17: The method of any preceding aspect, wherein the experiment or assay comprises at least one of: CTC genetic analysis for investigation of new and/or old mutations which drive tumor progression; CFC testing of genetic and/or chromosome abnormalities of an embryo; and counting CTC numbers in a given volume of blood.

Aspect 18: The method of any preceding aspect, wherein the experiment or assay comprises at least one of testing anti-cancer activity of one or more cancer therapies and/or agents; assessing target cell survival and/or growth; assessing target cell proliferation by measuring 3H-thymidine incorporation, by direct cell count, by detecting changes in transcriptional activity of known genes such as proto-oncogenes or cell cycle markers; assessing cell viability by trypan blue staining, assessing differentiation visually based on changes in morphology, evaluating prophylactic and/or therapeutic utility of combinatorial therapies disclosed herein for treatment, prophylaxis, management or amelioration of one or more symptoms associated with the disease or disorder as described hereinabove, and the like.

Aspect 19: The method of any preceding aspect, wherein the target cell is a mammalian cell.

Aspect 20: The method of any preceding aspect, wherein the mammalian cell is a human cell.

Aspect 21: The method of any preceding aspect, wherein the effective amount is a therapeutically effective amount.

Aspect 22: The method of any preceding aspect, wherein the effective amount is a prophylactically effective amount.

Aspect 23: The method of any preceding aspect, further comprising the step of identifying a target cell and/or subject in need of treatment for a disorder or disease of uncontrolled cellular proliferation.

Aspect 24: The method of any preceding aspect, wherein the isolated target cell and/or subject has been diagnosed with a need for treatment of a disorder or disease of uncontrolled cellular proliferation prior to the administering step.

Aspect 25: The method of any preceding aspect, wherein the target cell and/or subject in need of treatment comprises having at least one risk factor for developing a disorder or disease of uncontrolled cellular proliferation.

Aspect 26: The method of any preceding aspect, wherein diagnosing comprises performing an experiment, assay, and/or physical examination upon the subject and making a finding.

Aspect 27: The method of any preceding aspect, wherein the finding comprises identifying at least one risk factor for developing a disorder or disease of uncontrolled cellular proliferation.

Aspect 28: The method of any preceding aspect, wherein diagnosing comprises performing an experiment and/or assay upon the target cell and/or subject and identifying a level of a biological marker.

Aspect 29: The method of any preceding aspect, wherein the biological marker is a marker for a disorder or disease of uncontrolled cellular proliferation.

Aspect 30: The method of any preceding aspect, wherein the subject is a mammal.

Aspect 31: The method of any preceding aspect, wherein the mammal is a human.

Aspect 32: The method of any preceding aspect, where in the subject is a biological sample.

Aspect 33: The method of any preceding aspect, where in the biological sample is selected from a cell, blood, saliva, urine, tissue, or phlegm.

Aspect 34: The method of any preceding aspect, wherein the disorder or disease of uncontrolled cellular proliferation is a cancer, tumorigenesis, or malignant cancer development.

Aspect 35: The method of any preceding aspect, wherein the cancer is a hematological cancer.

Aspect 36: The method of any preceding aspect, wherein the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

Aspect 37: The method of any preceding aspect, wherein the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

Aspect 38: The method of any preceding aspect, wherein the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenström macroglobulinemia.

Aspect 39: The method of any preceding aspect, wherein the chronic myeloproliferative disorder is selected from chronic myelogenous leukemia, polycythemia vera, primary myelofibrosis, chronic idiopathic myelofibrosis, essential thrombocythemia, chronic neutrophilic leukemia, and chronic eosinophilic leukemia.

Aspect 40: The method of any preceding aspect, further comprising administering to the isolated target cell, biological fluid, and/or subject an effective amount of at least one disclosed cancer treatment and/or agent known to treat a disorder of uncontrolled cellular proliferation.

Aspect 41: The method of any preceding aspect, wherein selection of the at least one cancer treatment and/or agent is based on the experiment or assay.

Aspect 42: The method of any preceding aspect, wherein selection of the at least one cancer treatment comprises the use of at least one of radio waves, infrared, radiation, laser, and thermal ablation.

Aspect 43: The method of any preceding aspect, wherein the at least one agent is a hormone therapy agent. In a still further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

Aspect 44: The method of any preceding aspect, wherein the at least one agent is a chemotherapeutic agent. In a still further aspect, the chemotherapeutic agent is selected from one or more of the group consisting of an alkylating-like agent, an antimetabolite agent, an antineoplastic antibiotic agent, a mitotic inhibitor agent, an mTor inhibitor agent or other chemotherapeutic agent.

Aspect 45: The method of any preceding aspect, wherein the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.

Aspect 46: The method of any preceding aspect, wherein the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

Aspect 47: The method of any preceding aspect, wherein the alkylating-like agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

Aspect 48: The method of any preceding aspect, wherein the mitotic inhibitor agent is selected from one or more of the group consisting of etopside, vincristine, ixabepilone, vinorelbine, vinblastine, and teniposide, or a pharmaceutically acceptable salt, hydrate, solvate, or polymorph thereof.

Aspect 49: The method of any preceding aspect, wherein the mTor inhibitor agent is selected from one or more of the group consisting of everolimus, siroliumus, and temsirolimus, or a pharmaceutically acceptable salt, hydrate, solvate, or polymorph thereof.

Aspect 50: A composition comprising at least one iron saccharide complex or a pharmaceutically acceptable derivative thereof capable of generating a magnetically responsive intracellular concentration.

Aspect 51: The composition of any preceding aspect, wherein the at least one iron saccharide complex or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field.

Aspect 52: The composition of any preceding aspect, wherein the saccharide of the least one iron saccharide complex or a pharmaceutically acceptable derivative thereof comprises a monosaccharide, disaccharide, or polysaccharide, or a combination thereof.

Aspect 53: The composition of any preceding aspect, wherein a saccharide of the least one iron saccharide complex or a pharmaceutically acceptable derivative thereof comprises at least one of: glucose, dextrose, 2-deoxyglucose, thioglucose, galactose, fructose, dextran, dextrin, maltose, arabinose, rhamnose, mannose, sorbose, xylose, lactose, sucrose and raffmose.

Aspect 54: The composition of any preceding aspect, wherein the least one iron saccharide complex or a pharmaceutically acceptable derivative thereof comprises at least one of: iron glucose, iron 2-deoxyglucose, iron thioglucose, iron sucrose, iron dextran, iron fructose, polysaccharide iron complex (PIC) or the like.

Aspect 55: The composition of any preceding aspect, wherein the effective amount is a therapeutically effective amount.

Aspect 56: The composition of any preceding aspect, wherein the effective amount is a prophylactically effective amount.

Aspect 57: The composition of any preceding aspect, wherein the composition is formulated for oral administration.

Aspect 58: The composition of any preceding aspect, wherein the composition is formulated for parenteral administration.

Aspect 59: A kit comprising an effective amount of at least one iron saccharide complex or a pharmaceutically acceptable derivative thereof, and one or more of: a) instructions the iron saccharide complex in connection with isolating one or target cells associated with a disorder or disease of uncontrolled cellular proliferation using; b) at least one agent known to treat the disorder or disease of uncontrolled cellular proliferation; and c) instructions for treating the disorder or disease of uncontrolled cellular proliferation.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1) A method for isolating one or more target cells in a biological fluid of a subject, the method comprising: selectively providing to the one or more target cells an effective amount of at least one iron saccharide complex or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field.

2) The method of claim 1, wherein a saccharide of the least one iron saccharide complex or a pharmaceutically acceptable derivative thereof comprises a monosaccharide, disaccharide, or polysaccharide, or a combination thereof.

3) The method of claim 2, wherein a saccharide of the least one iron saccharide complex or a pharmaceutically acceptable derivative thereof comprises at least one of: glucose, dextrose, 2-deoxyglucose, thioglucose, galactose, fructose, dextran, dextrin, maltose, arabinose, rhamnose, mannose, sorbose, xylose, lactose, sucrose and raffmose.

4) The method of claim 2, wherein the least one iron saccharide complex or a pharmaceutically acceptable derivative thereof comprises at least one of: iron glucose, iron 2-deoxyglucose, iron thioglucose, iron sucrose, iron dextran, iron fructose, and polysaccharide iron complex (PIC).

5) The method of claim 4, wherein the one or more target cells are at least one of circulating tumor cells (CTCs), circulating fetal cells (CFCs), and circulating stem cells (CSCs).

6) The method of claim 5, wherein isolating comprises passing the biological fluid containing the one or more target cells through a magnetic separation device or magnetic filter.

7) The method of claim 6, wherein the magnetic filter comprises a venous circulation or arterio-venous shunt.

8) The method of claim 7, wherein isolating comprises passing the biological fluid containing the target cells through a microfluidic magnetic separation device.

9) The method of claim 8, wherein providing comprises introducing into the one or more target cell a magnetically responsive amount of the at least one iron saccharide complex.

10) The method of claim 9, wherein providing comprises incubating the target cell in a preparation containing the at least one iron saccharide complex and pharmaceutical compositions comprising same for a predetermined time.

11) The method of claim 10, wherein isolating the one or more target cells comprises at least one of:
a) contacting the biological fluid with a magnetic separation device, the magnetic separation device comprising a magnetic source and a tube;
b) producing a continuous relative motion between the magnetic source and the biological fluid while the magnetic source produces a magnetic field across the tube, such that magnetically responsive target cells are selectively captured against the tube and wherein the continuous relative motion is applied such that a majority of the biological fluid has access to the magnetic source; and
c) substantially removing the magnetic field produced by the magnetic source across the tube; wherein the magnetically responsive target cells are released from the tube wall.

12) The method of claim 11, wherein the biological fluid comprises at least one of body fluid, whole blood, plasma, any cell-containing blood fraction, cerebrospinal fluid, bone marrow, cell sample, saliva, urine, feces, tissue, or phlegm; and further comprising performing an experiment or assay on at least one of: the isolated target cells, biological fluid, and the subject.

13) The method of claim 12, wherein the experiment or assay comprises at least one of identification of a level of a biological marker, cell culture, an immunochemical analysis, morphological analysis, genomics analysis, metabolomic-s, epigenomics analysis, proteomics analysis, DNA mutation analysis, whole genome analysis, protein and RNA expression level of a specific gene or a combination thereof.

14) The method of claim 13, wherein the experiment or assay comprises at least one of: CTC genetic analysis for investigation of new or old mutations which drive tumor progression; CFC testing of genetic or chromosome abnormalities of an embryo; and counting CTC numbers in a given volume of blood.

15) The method of claim 14, wherein the experiment or assay comprises at least one of testing anti-cancer activity of one or more cancer therapies or agents; assessing target cell survival or growth; assessing target cell proliferation by measuring 3H-thymidine incorporation, by direct cell count, by detecting changes in transcriptional activity of known genes such as proto-oncogenes or cell cycle markers; assessing cell viability by trypan blue staining, assessing differentiation visually based on changes in morphology, evaluating prophylactic or therapeutic utility of combinatorial therapies disclosed herein for treatment, prophylaxis, management or amelioration of one or more symptoms associated with the disease or disorder, or combinations thereof.

16) The method of claim 15, further comprising administering to the isolated target cell, biological fluid, or subject an effective amount of at least one cancer treatment or agent known to treat a disorder of uncontrolled cellular proliferation; and wherein a selection of the at least one cancer treatment or agent is based on the experiment or assay.

17) The method of claim 16, wherein selection of the at least one cancer treatment comprises the use of at least one of radio waves, infrared, radiation, laser, and thermal ablation.

18) A composition comprising at least one iron saccharide complex or a pharmaceutically acceptable derivative thereof capable of generating a magnetically responsive intracellular concentration; wherein the at least one iron saccharide complex or a pharmaceutically acceptable derivative thereof; and isolating the target cells using a magnetic source capable of generating a magnetic field effective to capture target cells containing a magnetically responsive intracellular concentration of the of at least one iron saccharide complex or a pharmaceutically acceptable derivative thereof in the magnetic field.

19) The composition of claim 18, wherein the saccharide of the least one iron saccharide complex or a pharmaceutically acceptable derivative thereof comprises a monosaccharide, disaccharide, or polysaccharide, or a combination thereof.

20) The composition of claim 19, wherein a saccharide of the least one iron saccharide complex or a pharmaceutically acceptable derivative thereof comprises at least one of: glucose, dextrose, 2-deoxyglucose, thioglucose, galactose, fructose, dextran, dextrin, maltose, arabinose, rhamnose, mannose, sorbose, xylose, lactose, sucrose and raffmose.
